(19) United States
(12) Patent Application Publication
(10) Pub. No.: US 2015/0075699 A1
(43) Pub. Date: Mar. 19, 2015

(54) Method of Forming a Prosthesis Using a Hollow Exterior Shell

(71) Applicant: Lifeart Prosthetics Inc., West St. Paul (CA)

(72) Inventor: Wendy Smith, West St. Paul (CA)

(21) Appl. No.: 14/306,329

(22) Filed: Jun. 17, 2014

Related U.S. Application Data
(60) Provisional application No. 61/837,282, filed on Jun. 20, 2013.

(51) Int. Cl. A61F 2/12

(52) U.S. Cl. CPC A61F 2/12; A61F 2240/001; A61F 2240/004
USPC 156/61

(57) ABSTRACT

A prosthesis is formed by providing a first positive mold portion representation of a prosthetic mating body portion of the user and a second positive mold portion representing a symmetrical body portion of the user. In one instance a front shell portion is molded overtop of the second positive mold portion and a rear shell portion is positioned over the first positive mold portion prior to joining with the application of a finish coating of silicone. In some instances a fabric material is stretched onto one of the mold portions prior to coating the fabric with silicone such that the fabric is impregnated with silicone to define an exterior shell of the prosthesis once cured. In a further instance, a resilient prosthetic shell is formed using negative molds of the prosthetic mating and symmetrical body portions of the user prior to filling the shell with a core material.

This application claims the benefit under 35 U.S.C. 119(e) of U.S. provisional application Ser. No. 61/837,282, filed Jun. 20, 2013.

### FIELD OF THE INVENTION

The present invention relates to a method of forming an external prosthesis, for example a breast prosthesis, and more particularly the present invention relates to a method of molding an exterior shell of the prosthesis prior to filling with a core material.

### BACKGROUND

Various forms of external breast prostheses exist in the prior art and use of external breast prosthesis following a mastectomy for example is well known. Conventional manufacturing techniques are generally considered to either: i) be uncomfortable; ii) not fit properly; and iii) most importantly, lack the ability to be customized to a particular user in an efficient and cost effective manner.

### SUMMARY OF THE INVENTION

According to one aspect of the invention there is provided a method of forming a prosthesis for mating with a first body portion of a user such that the prosthesis is symmetrical with a second body portion of the user, the method comprising:

providing a first positive mold portion which is representative of the first body portion of the user;

providing a second positive mold portion which is representative of the second body portion of the user;

molding a front shell portion overtop of the second positive mold portion;

positioning a rear shell portion on the first positive mold portion;

joining the molded front shell portion to the rear shell portion on the first positive mold portion by applying a finish coating of silicone in an uncured state to a front side of the shell portions and allowing the silicone to cure such that the front shell portion defines a front side of an exterior shell of the prosthesis which is symmetrical with the second body portion of the user and rear shell portion defines a rear side of the exterior shell of the prosthesis which mates with the first body portion of the user.

Preferably one or both of the front and rear shell portions comprises a fabric, or more particularly a silicone infused fabric material. The silicone infused fabric material may be formed by shaping a fabric material overtop of the mold portion, coating the fabric with silicone such that the fabric is impregnated with the silicone, and maintaining a shape of the fabric material on the mold portion until the silicone has cured.

More particularly, the front shell portion may be molded using silicone foam material.

The method may further include positioning the rear shell portion on the first positive mold portion by positioning two sheet panels on the first positive mold portion to overlap one another along a seam such that the seam spans across the rear side of the resulting prosthesis for accessing a hollow interior of the prosthesis therethrough.

The method may further include trimming a perimeter edge of the front shell portion subsequent to the front shell portion being molding on the second positive mold portion and prior to joining to the rear shell portion.

Preferably the hollow interior of the exterior shell is filled subsequent to the front and rear shell portions being joined to form the exterior shell.

Preferably a vent aperture is provided in communication through the exterior shell such that a hollow interior of the exterior shell is externally vented therethrough. The vent is preferably formed at a perimeter edge between the front and rear shell portions as they are joined.

The vent aperture may be formed by placing a vent core between the front and rear shell portions prior to joining the front and rear shell portions in which the vent core comprises an open cell foam defining a passageway in communication with the hollow interior of the exterior shell.

According to a second aspect of the present invention there is provided a method of forming at least a portion of a prosthesis for mating with a first body portion of a user such that the prosthesis is symmetrical with a second body portion of the user, the method comprising:

providing a positive mold portion which is representative of at least one body portion of the user;

shaping a fabric material overtop of said at least one positive mold portion;

coating the fabric with silicone such that the fabric is impregnated with silicone; and

maintaining a shape of the fabric material overtop of the second positive mold portion until the silicone has cured to define at least a portion of an exterior shell of the prosthesis.

When the positive mold portion has a front side which is representative of the second body portion of the user, preferably the fabric is shaped to extend overtop of at least the front side of the positive mold portion.

The method may further include trimming the silicone impregnated fabric about a perimeter edge such that the silicone impregnated fabric defines only a front side of an exterior shell of a resulting prosthesis.

When the positive mold portion also includes a rear side which is arranged to mate with the first body portion of the user, the method may further include shaping the fabric to extend overtop of the rear side of the positive mold portion such that the silicone impregnated fabric substantially fully defines an exterior shell of a resulting prosthesis.

When a stem is connected to the positive mold portion at a perimeter edge between the front and rear sides preferably the stem is used to define the vent aperture in exterior shell.

The method may further include providing an aperture in the exterior shell and removing the positive mold portion from the exterior shell through the aperture.

In some embodiments, the exterior shell may be inverted prior to filling a hollow interior with a core material.

According to a further aspect of the present invention there is provided a method of forming a prosthesis for mating with a first body portion of a user such that the prosthesis is symmetrical with a second body portion of the user, the method comprising:

providing a first negative mold portion which is representative of the first body portion of the user;

providing a second negative mold portion which is arranged to mate with a symmetrical reproduction of the second body portion of the user;

joining the first and second negative mold portions to form a rotational mold having a vent aperture therein;

placing a molding material in the rotation mold through the vent aperture;

rotating the mold to coat the first and second negative mold portions with the molding material;

allowing the molding material to cure to form an exterior shell;

removing the exterior shell from the rotational mold; and

filling a hollow interior of the exterior shell with a core material subsequent to removal from the rotational mold.

Preferably the vent aperture in this instance is located at a perimeter edge define at a junction of the first and second negative mold portions.

The method may further include filling the hollow interior with the core material by injecting the core material through an aperture in the exterior shell formed at the vent aperture.

In each instance, the mold portions can be custom fitted and derived from the actual user of the prosthesis, or can be derived from a finite set of predefined shapes and sizes so as to readily vary the amount of customization desired. Only the exterior shell requires customization, but can be readily filled with a core material according to user preference depending upon desired use. The construction is also well suited to the core being provided as a set of interchangeable modular elements.

Various embodiments of the invention will now be described in conjunction with the accompanying drawings in which:

### BRIEF DESCRIPTION OF THE DRAWINGS

FIG. 1 is a schematic representation of an exemplary prosthesis according to the present invention;

FIG. 2 is a section view along the line 2-2 of FIG. 1;

FIG. 3 is a schematic representation of the molding steps of the first and second mold portions directly from a user in a fully customized application;

FIG. 4 is a schematic representation of the steps in forming a prosthesis according to a first embodiment;

FIG. 5 is a flow chart representing the steps in forming the prosthesis according to FIG. 4;

FIG. 6 is a schematic representation of the steps in forming a prosthesis according to a second embodiment;

FIG. 7 is a flow chart representing the steps in forming the prosthesis according to FIG. 6;

FIG. 8 is a schematic representation of the steps in forming a prosthesis according to a third embodiment; and

FIG. 9 is a flow chart representing the steps in forming the prosthesis according to FIG. 8.

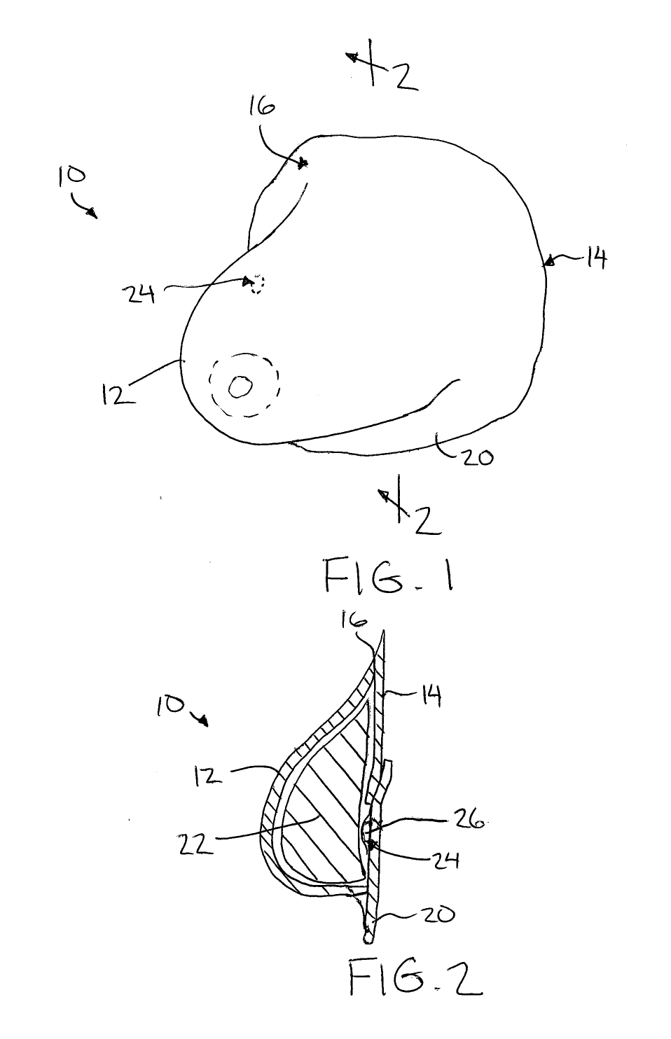

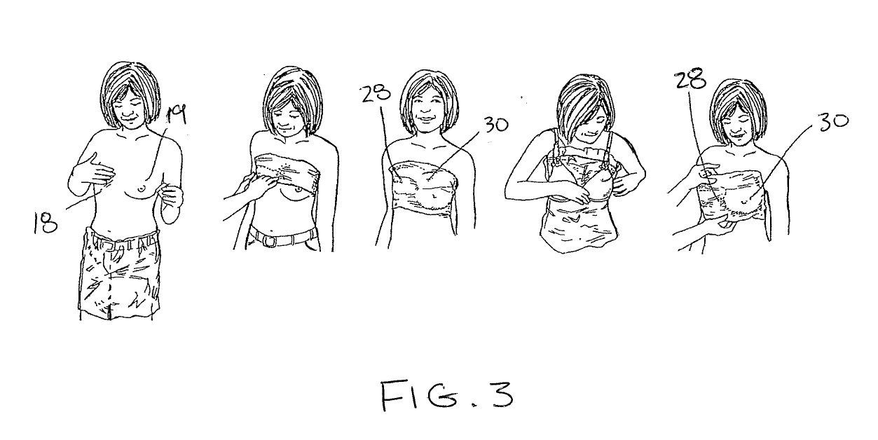

In the drawings like characters of reference indicate corresponding parts in the different figures.

### DETAILED DESCRIPTION

Referring to the accompanying figures there is illustrated various embodiments of a breast prosthesis generally indicated by reference numeral 10. The prosthesis 10 is typically formed of a shell having a front side 12 and a rear side 14 joined to one another at a perimeter edge 16. The rear side 14 is arranged to mate with a first body portion 18 of a user which corresponds to the chest area of a patient being fitted for a breast prosthesis. The front side 12 corresponds to the protruding portion which protrudes outwardly from the chest area of the patient so as to be symmetrical with a corresponding second body portion 19 of the user which in the instance of a patient being fitted for a breast prosthesis corresponds to the other breast of the patient.

The prosthesis is typically shaped so that the front side tapers gradually into the plane of the rear side at the top end for gradually blending into the surrounding skin of the user. An underband flange 20 is provided along the bottom perimeter edge in the form of a flange line generally in a common plane with the plane of the rear side of the prosthesis so as to be suitably positioned for being overlapped by a bra of the patient for securement of the prosthesis in place in use.

A core material 22 typically fills the hollow interior of the shell between the front and rear sides such that the core material is fully enclosed between the front and rear sides of the shell. The core material is typically filled into the shell after the shell has been formed and the front and rear sides have already been joined at the perimeter edge.

In many instances the prosthesis further comprises a vent 24 in the form of a passageway in communication through the front side of the shell between interior and exterior sides thereof at a location adjacent the perimeter edge between the front and rear sides. The vent 24 is typically formed by a foam core 26 comprising open celled foam positioned between the front and rear sides prior to joining the front and rear sides such that the foam maintains the passageway of the vent open between interior and exterior sides as the front and rear of the prosthesis are joined together.

The prosthesis is typically formed by initially providing two separate mold portions. The mold portions include a first mold portion 28 corresponding to the first body portion of the user, that is the chest area of the patient for forming the rear side of the prosthesis. The second mold portion 30 corresponds to a symmetrical reproduction of the other body part with which the prosthesis must be symmetrical for forming the front side of the prosthesis. The initial mold portions comprise negative molds arranged to mate with the respective first and second body portions of the user respectively and are formed by taking actual castings from the body of the user as shown in FIG. 3. In alternative arrangements the initial molds can be derived from a finite set of preshaped and presized molds which are derived from actual measurements from previously fitted woman.

Some steps in the following methods require a positive mold which is formed by joining the first and second mold portions 28 and 30 and filling the resulting mold so that the formed part which forms the positive mold is representative of the actual prosthesis, that is the breast that the prosthesis is intended to replicate.

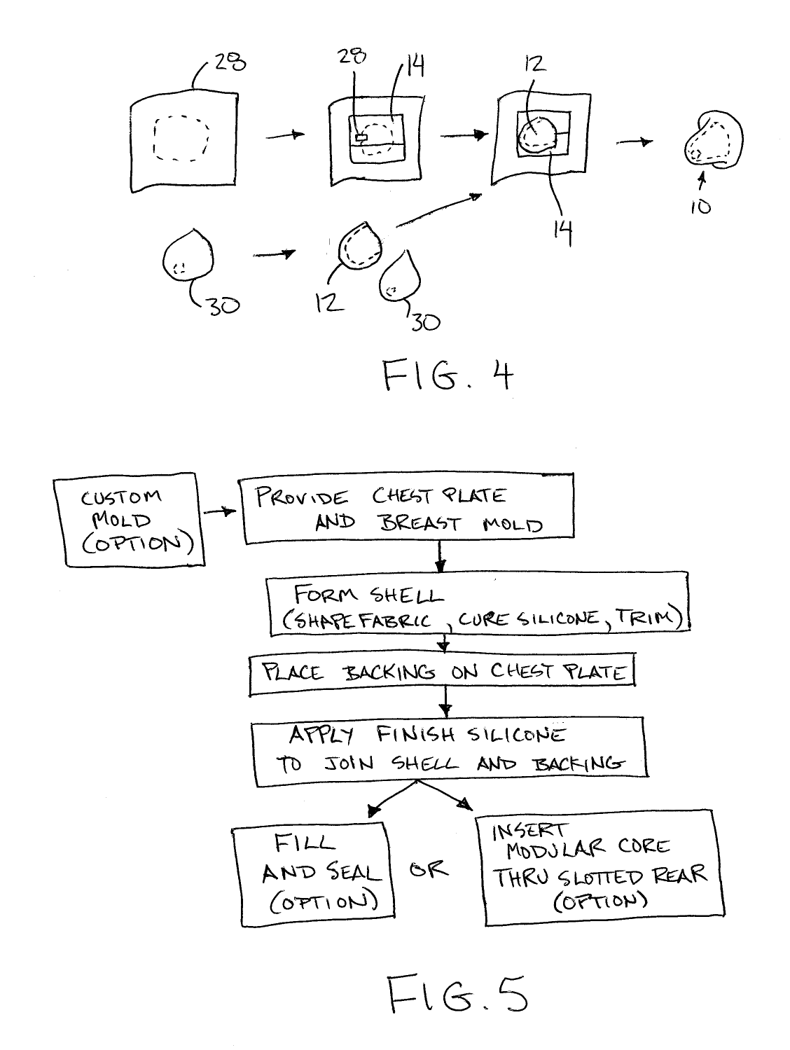

Turning now more particularly to FIGS. 4 and 5, the first method of forming the prosthesis will now be described in further detail. In this instance the manufacturing process starts by providing a chest plate formed from the first mold portion 28 and a breast mold formed from the second mold portion 30. The breast mold is a positive mold in this instance having the likeness of the actual finished prosthesis. The chest plate is prepared by placing material forming the rear side of the prosthesis over the corresponding mold portion of the chest plate. This may be accomplished by initially laying a fabric material over the chest plate to be subsequently coated with silicone which impregnates the fabric for curing in place on the chest plate.

Alternatively a pre-manufactured silicone sheet or fabric sheet impregnated with silicone can be laid over the chest plate to form the backing.

In yet a further arrangement two sheets of pre-manufactured silicone impregnated fabric are laid over top one another to form a seam of overlapping sheets in the rear side of the subsequently manufactured prosthesis which provides access to the hollow interior for inserting and removing core material from the shell as desired. The foam core 26 for forming the vent is also placed at the perimeter location of the prosthesis to be formed.

The front side of the prosthesis is then formed using the breast mold by stretching or shaping a fabric overtop of the breast mold and coating the fabric with silicone such that the silicone impregnates the fabric. The fabric is maintained in the desired shape over the positive breast mold until the poured silicone has set. In a preferred embodiment a silicone foam is used so as to remain lightweight yet durable due to the composite fabric construction. The resulting molded article is trimmed about its perimeter edge and removed from the positive breast mold for subsequent placement overtop of the previously prepared backing layers on the chest plate.

The front and rear sides of the molded and cured silicone material are then joined together by applying more silicone in an uncured state overtop of the front side and forward facing face of the rear side protruding radially outward beyond the front side at the top and bottom ends so that the subsequent silicone being applied forms both the outer finished coat of the finished prosthesis as well as provides structural bonding between the front and rear sides. The underband and tapered edge at the top of the finished prosthesis may be formed by the finishing layers of silicone being applied or may be formed in part with the backing which defines the rear side of the finished prosthesis.

The hollow interior of the shell can then be filled with core material at one time at the manufacturing step with the shell being subsequently sealed closed to form a first form of the finished prosthesis which is typically associated with minimal customization to the user. The rear side typically comprises a continuous sheet in this instance.

In an alternative arrangement when providing access using an overlapping flap seam in the rear side, the hollow interior can be filled with a pre-manufactured and shaped foam core or other soft resilient materials for example to provide some firmness to the finished shape of the exterior shell without adding considerable weight. Different configurations of cores may be provided for interchanging use depending upon the desired activity of the user in use. In this instance the prosthesis may also be formed using custom mold portions fitted to the user or standardized sized mold portions.

In yet a further arrangement both the front and rear sides are formed using custom shaped mold portions fitted to the user and other attention to detail is provided in the finished coat where coloring is applied to ensure a custom application of coloring and details such as veins, skin color, areola, tattoos or other options selected by a user.

Once the front and rear side have been joined together with the finished coats, or prior to the application of all finished coats, the excess backing material can be trimmed to form the finished dimensions of the rear side of the prosthesis and the prosthesis can be removed from the chest plate.

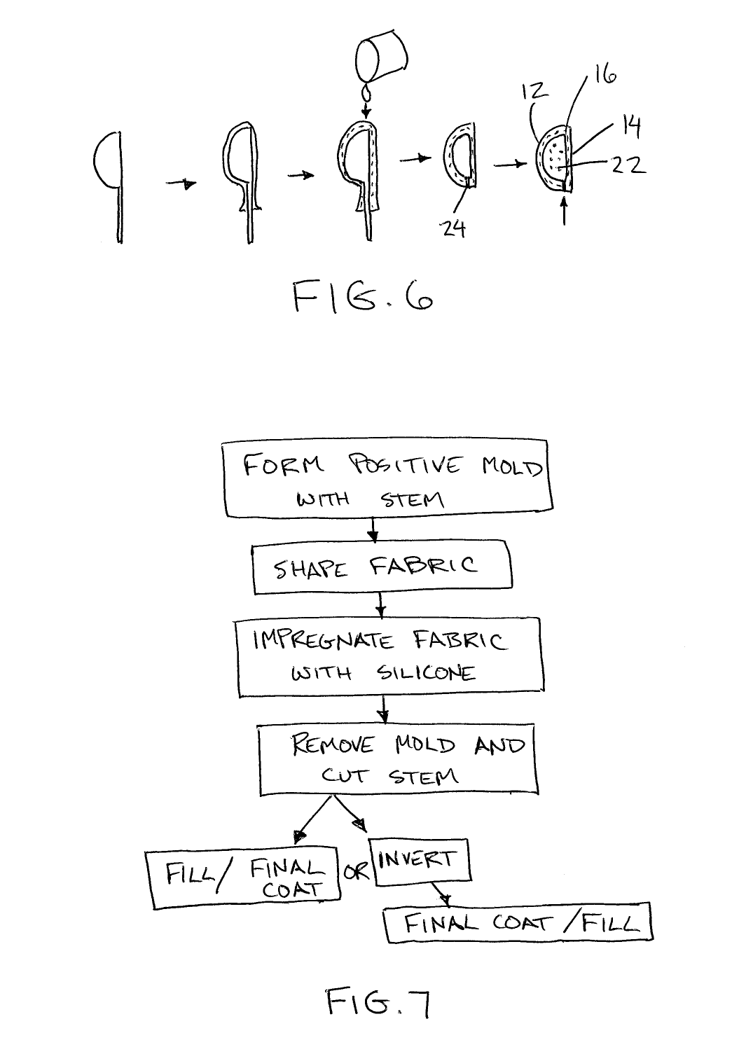

Turning now to FIGS. 6 and 7, according to a further embodiment, a positive breast mold is again formed using the first and second mold portions, however in this instance an additional stem is attached at the perimeter edge corresponding to the desired vent location. A suitable fabric material is again stretched and shaped overtop of the positive breast mold and silicone material is coated overtop of the fabric such that the silicone impregnates the fabric about both the front and rear sides of the positive breast mold. The silicone is preferably a silicone foam which is lightweight yet maintains strength due to the fabric impregnated therein. Once the silicone has cured on the breast mold, the stem of the mold can be cut off and the molded shell can be removed from overtop of the positive breast mold.

In one example the rear side is cut to form a slot spanning the width of the rear side for access to remove the mold and replace the mold with a suitable core of any desired material.

In a further instance the material is stretched from the vent aperture corresponding to the stem location at the perimeter edge to permit removal of the positive mold from the interior of the shell by insertion through the resulting aperture formed by the stem. The core material in this instance can be inserted into the resulting hollow interior through the same aperture either by filling with a foam material to be cured in place or by inserting an already formed core and stretching the shell over the core.

In yet further instances the shell formed on the positive breast mold can be removed by either methods of forming a slot in the rear side or removal through the stem aperture but an additional step of reversing the mold is provided prior to filling the resulting hollow interior with fill material forming the core. In all instances a final coat may or may not be applied to the resulting article molded on the positive mold depending upon the desired application. In the instance of a swim prosthesis for instance the core is typically filled through the stem aperture with the shell being either in its molded state or in a reversed state. The stem aperture is then sealed closed after filling the core material so that the resulting fill forms a waterproof bag enclosure.

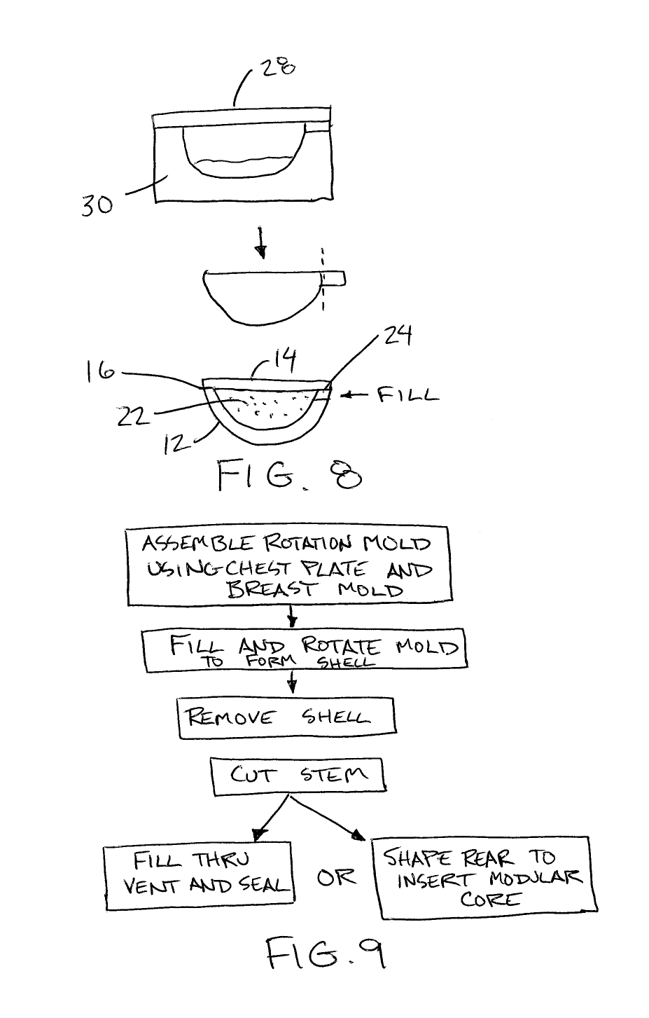

Turning now to the embodiment of FIGS. 8 and 9, in this instance the first and second mold portions derived from the first and second body portions of the user are joined together to form a negative mold with a vent of the mold be located at the perimeter edge between the two mold portions. Uncured silicone material, preferably silicone foam can then be inserted into the mold through the vent opening sufficient in quantity only to coat the interior of the mold as the mold is rotated for rotationally molding a resulting shell of silicone within the negative mold. Once cured the molded article is removed from the mold and the resulting stem which is integrally molded within the vent passage of the mold can be cut off to form the desired finished shape of the breast prosthesis. Foam core material may again be located within the vent passageway to be molded in place between the front and rear sides of the prosthesis. Preferably the passageway remains open however so that the core material can be subsequently filled by insertion through the resulting vent aperture of the mold once the molded article has been removed from the mold and the stem has been cut. Once the core material has been filled into the mold the vent opening can be sealed closed or can remain open and define the resulting vent in the finished prosthesis at the perimeter edge between the front and rear sides.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A method of forming a prosthesis for mating with a first body portion of a user such that the prosthesis is symmetrical with a second body portion of the user, the method comprising:
providing a first positive mold portion which is representative of the first body portion of the user;
providing a second positive mold portion which is representative of the second body portion of the user;
molding a front shell portion overtop of the second positive mold portion;
positioning a rear shell portion on the first positive mold portion;
joining the molded front shell portion to the rear shell portion on the first positive mold portion by applying a finish coating of silicone in an uncured state to a front side of the shell portions and allowing the silicone to cure such that the front shell portion defines a front side of an exterior shell of the prosthesis which is symmetrical with the second body portion of the user and rear shell portion defines a rear side of the exterior shell of the prosthesis which mates with the first body portion of the user.

2. The method according to claim 1 wherein the rear shell portion comprises a fabric material.

3. The method according to claim 1 wherein the rear shell portion comprises a silicone infused fabric material.

4. The method according to claim 1 positioning the rear shell portion on the first positive mold portion further includes positioning two sheet panels on the first positive mold portion to overlap one another along a seam such that the seam spans across the rear side of the resulting prosthesis for accessing a hollow interior of the prosthesis therethrough.

5. The method according to claim 1 including molding the front shell portion using silicone foam material.

6. The method according to claim 1 including molding the front shell portion by shaping a fabric material overtop of the second positive mold portion, coating the fabric with silicone such that the fabric is impregnated with silicone, and maintaining a shape of the fabric material overtop of the second positive mold portion until the silicone has cured.

7. The method according to claim 1 including trimming a perimeter edge of the front shell portion subsequent to being molding on the second positive mold portion and prior to joining to the rear shell portion.

8. The method according to claim 1 including filling a hollow interior of the exterior shell subsequent to the front and rear shell portions being joined to form the exterior shell.

9. The method according to claim 1 including forming a vent aperture in communication through the exterior shell such that a hollow interior of the exterior shell is externally vented therethrough.

10. The method according to claim 9 including forming the vent aperture by placing a vent core between the front and rear shell portions prior to joining the front and rear shell portions in which the vent core comprises an open cell foam defining a passageway in communication with the hollow interior of the exterior shell.

11. A method of forming a portion of a prosthesis for mating with a first body, portion of a user such that the prosthesis is symmetrical with a second body portion of the user, the method comprising:
providing a positive mold portion which is representative of at least one body portion of the user;
shaping a fabric material overtop of said at least one positive mold portion;
coating the fabric with silicone such that the fabric is impregnated with silicone; and
maintaining a shape of the fabric material overtop of the second positive mold portion until the silicone has cured to define at least a portion of an exterior shell of the prosthesis.

12. The method according to claim 11 including providing the positive mold portion with a front side which is representative of the second body portion of the user, and shaping the fabric to extend overtop of at least the front side of the positive mold portion.

13. The method according to claim 12 including trimming the silicone impregnated fabric about a perimeter edge such that the silicone impregnated fabric defines only a front side of an exterior shell of a resulting prosthesis.

14. The method according to claim 12 including providing the positive mold portion with a rear side which is arranged to mate with the first body portion of the user, and shaping the fabric to extend overtop of the rear side of the positive mold portion such that the silicone impregnated fabric substantially fully defines an exterior shell of a resulting prosthesis.

15. The method according to claim 14 including providing a stem connected to the positive mold portion at a perimeter edge between the front and rear sides about which the fabric is shaped and the silicone is provided so as to define a vent aperture through the exterior shell in communication with a hollow interior of the exterior shell.

16. The method according to claim 14 including providing an aperture in the exterior shell and removing the positive mold portion from the exterior shell through the aperture.

17. The method according to claim 11 including inverting the exterior shell prior to filling a hollow interior with a core material.

18. A method of forming a prosthesis for mating with a first body portion of a user such that the prosthesis is symmetrical with a second body portion of the user, the method comprising:
providing a first negative mold portion which is representative of the first body portion of the user;
providing a second negative mold portion which is arranged to mate with a symmetrical reproduction of the second body portion of the user;
joining the first and second negative mold portions to form a rotational mold having a vent aperture therein;
placing a molding material in the rotation mold through the vent aperture;
rotating the mold to coat the first and second negative mold portions with the molding material;
allowing the molding material to cure to form an exterior shell;
removing the exterior shell from the rotational mold; and
filling a hollow interior of the exterior shell with a core material subsequent to removal from the rotational mold.

19. The method according to claim 18 including locating the vent aperture at a perimeter edge define at a junction of the first and second negative mold portions.

20. The method according to claim 18 including filling the hollow interior with the core material by injecting the core material through an aperture in the exterior shell formed at the vent aperture.
